(19) United States
(12) Patent Application Publication
(10) Pub. No.: US 2007/0213406 A1
(43) Pub. Date: Sep. 13, 2007

(54) Anti-Inflammatory Analgesic External Aqueus Liquid Preparation

(75) Inventors: Yutaka Kawamura, Narita-shi (JP); Itsuro Fujita, Fukushima-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: DOJIN IYAKU-KAKO CO., LTD, TOKYO (JP)

(21) Appl. No.: 11/578,152

(22) PCT Filed: Mar. 23, 2005
(86) PCT No.: PCT/JP05/09337

§ 371 Date: Oct. 11, 2006

(30) Foreign Application Priority Data
May 24, 2004 (JP) 2004-153657

(51) Int. Cl. A61K 31/196; A61K 47/10; A61K 47/14; A61K 47/38; A61K 9/08; A61P 29/00

(52) U.S. Cl. 514/658

(57) ABSTRACT

An anti-inflammatory analgesic external aqueous liquid preparation that excels in percutaneous absorbability and applicability, and being stable over time. There is provided an anti-inflammatory analgesic external aqueous liquid preparation comprising diclofenac or its salt, a dibasic acid ester, a lower alcohol, and 0.05 to 0.45 wt % of thickening agent.

### TECHNICAL FIELD

The present invention relates to an anti-inflammatory analgesic external aqueous liquid preparation excellent in percutaneous absorbability and applicability, being stable over time.

### BACKGROUND ART

Diclofenac and its salt have an excellent anti-inflammatory and analgesic effect, and are widely used clinically as oral preparation or suppository. However, when used as oral preparation or suppository, it is known to cause various side effects such as a gastrointestinal tract disorder. To alleviate such side effects, it is proposed to use an external application to have local or systemic effect by percutaneous absorption without passing through gastrointestinal tract. As external application targeted to manifestation of local effect, gel preparations using nonionic polymer (patent documents 1, 2) have been developed and put on market since 2000.

The gel preparation is excellent in percutaneous absorbability, but is high in viscosity because it is gel preparation, and since it cannot be contained in an application container having ball or foamed rubber at the top, it is required to be applied by hand. Some problems accompanying the gel preparation include longer time required for drying when applied on the skin, and further, if rubbed repeatedly in order to quicken the drying time, medical ingredients are lifted up.

It has hence been demanded to develop an external preparation easy to use, quick in drying time, excellent in applicability, and effective at smaller dose than gel preparation.

[Patent document 1] WO 92/07561

[Patent document 2] JP-A-7-173058

### DISCLOSURE OF THE INVENTION

### Problem to be Solved by the Invention

However, an external aqueous liquid preparation comprising diclofenac or its salt as active ingredient, and having a dibasic acid ester blended in lower alcohol aqueous base as percutaneous absorption enhancer of diclofenac is highly effective as anti-inflammatory analgesic preparation, but is very unstable, and ingredients may be separated and precipitate even at room temperature.

It is hence an object of the invention to present an anti-inflammatory analgesic external aqueous liquid preparation excellent in percutaneous absorbability and applicability, being stable over time.

### MEANS FOR SOLVING THE PROBLEM

Under such circumstances, the inventors studied intensively to find that an anti-inflammatory analgesic external aqueous liquid preparation excellent in percutaneous absorbability and applicability, and is stable over time can be obtained by blending diclofenac or its salt, a dibasic acid ester, a lower alcohol, and a specific amount of thickening agent, and have thereby completed the invention.

That is, the invention provides an anti-inflammatory analgesic external aqueous liquid preparation comprising diclofenac or its salt, a dibasic acid ester, a lower alcohol, and 0.05 to 0.45 wt % of thickening agent.

### EFFECT OF THE INVENTION

The anti-inflammatory analgesic external aqueous liquid preparation of the invention is excellent in percutaneous absorbability and applicability, stable over time, can exhibit the pharmacological action of diclofenac or its salt sufficiently, and has excellent anti-inflammatory and analgesic effects.

### BEST MODE FOR CARRYING OUT THE INVENTION

Diclofenac or its salt used in the invention is not particularly specified as long as it is pharmaceutically acceptable, and may include, for example, salts with alkali metals such as sodium and potassium; alkaline earth metals such as calcium and magnesium; ammonium; primary, secondary, or tertiary alkylamines such as dimethylamine, diethylamine, trimethylamine and triethylamine; and alkanolamines such as monoethanolamine, diisopropanolamine, diethanolamine, triethanolamine and triisopropanolamine. In particular, as salt of diclofenac, sodium salt and ammonium salt are preferred.

As diclofenac or its salt, diclofenac sodium and diclofenac ammonium are preferred.

Content of diclofenac or its salt in anti-inflammatory analgesic external aqueous liquid preparation is not particularly specified as long as the pharmacological effect is expressed, and generally it is 0.1 to 20 wt % or less (hereinafter indicated merely as %), preferably 0.1 to 15%, and more preferably 0.5 to 10%.

Dibasic acid ester is dissolved in mixed solution of lower alcohol and water, and is intended to enhance the skin penetration of diclofenac or its salt. Examples thereof include monovalent alcohol ester of 1 to 12 carbon atoms (preferably 2 to 8 carbon atoms) of dicarboxylic acid of 2 to 12 carbon atoms (preferably 2 to 8 carbon atoms). Preferable examples of dibasic acid ester include ester of adipic acid, succinic acid, or sebacic acid, and further include diisopropyl adipate, diethyl sebacate, diisopropyl sebacate, and dioctyl succinate, and diisopropyl adipate, diethyl sebacate, and diisopropyl sebacate are particularly preferred.

Dibasic acid ester is used either alone or in combination of two or more types.

Content of dibasic acid ester in anti-inflammatory analgesic external aqueous liquid preparation should be enough to absorb the desired diclofenac or its salt, and generally it is 0.5 to 20%, preferably 0.5 to 15%, and more preferably 1.5 to 10%.

Lower alcohol is not particularly specified as long as it is pharmaceutically acceptable. Examples of lower alcohol include monovalent alcohol of 1 to 5 carbon atoms, preferably ethyl alcohol, isopropyl alcohol, or a mixture thereof.

Content of lower alcohol in anti-inflammatory analgesic external aqueous liquid preparation varies depending on the type of thickening agent to be used together, pH of the aqueous liquid preparation, and type and amount of diclofenac or its salt and other liquid ingredients, and generally it is 10 to 80%, preferably 15 to 75%, and more preferably 25 to 70%.

Thickening agent is preferably water-soluble polymer, and examples thereof include methyl cellulose, hydroxy methyl cellulose, hydroxy ethyl cellulose, hydroxy propyl cellulose, hydroxy butyl cellulose, hydroxy ethyl methyl cellulose, hydroxy propyl methyl cellulose, polyvinyl pyrrolidone, and polyvinyl alcohol. Thickening agent may be used either alone or in combination of two or more types.

As thickening agent, in particular, hydroxy propyl cellulose and hydroxy ethyl cellulose are preferred. Commercially available products may be used, and hydroxypropyl cellulose includes MF (manufactured by Hercules) and HF (manufactured by Hercules), or hydroxy ethyl cellulose includes CF-W (manufactured by Fuji Chemical) and CF-X (manufactured by Fuji Chemical).

Content of thickening agent in anti-inflammatory analgesic external aqueous liquid preparation is 0.05 to 0.45%, and preferably 0.1 to 0.3%. If less than 0.05%, the aqueous liquid preparation is likely to cause dripping, and if exceeding 0.45%, the viscosity of aqueous liquid preparation is too high, the sense of use is inferior, and it is hard to obtain a clear aqueous liquid preparation.

If the pH of the anti-inflammatory analgesic external aqueous liquid preparation of the invention is at acid or alkali side, when the aqueous liquid preparation is applied repeatedly at the same area of the skin, percutaneous absorbability of diclofenac or its salt may be changed, and adverse effects such as skin irritation may be caused. Further, since the solubility of diclofenac or its salt depends on the pH of solvent, it may deposit crystals depending on changes of pH in the time course. Accordingly, in the anti-inflammatory analgesic external aqueous liquid preparation of the invention, it is preferred to add a pH regulator to adjust the pH to 5 to 8.5, and preferably 5.5 to 8. Herein, the value of pH is measured by a glass electrode type hydrogen ion concentration meter.

The material of pH regulator is not particularly specified as long as the pH of the aqueous liquid preparation can be adjusted to the above-specified range, and examples thereof include inorganic pH regulators such as sodium hydroxide, potassium hydroxide and hydrochloric acid; organic acids such as acetic acid, lacticacid, citricacid, malicacid, tartaricacid, maleic acid, fumaricacid, adipicacid, salicylicacid, or salts thereof, and they may be used either alone or in combination of two or more types. Further, a buffering action may be obtained by using an acidic pH regulator and basic pH regulator.

The method of manufacturing the anti-inflammatory analgesic external aqueous liquid preparation of the invention is not particularly specified, but generally it is prepared by dissolving thickening agent in purified water, mixing liquid ingredients, dissolving diclofenac or its salt therein, adjusting pH by adding pH regulator, and producing aqueous liquid preparation. However, it may be also prepared by other methods depending on the formulation of the aqueous liquid preparation or characteristic of pharmaceutical manufacturing equipment.

The anti-inflammatory analgesic external aqueous liquid preparation of the invention may also contain, as needed, moisturizing agent, co-solvent, stabilizer, odoring agent, and coloring agent in addition to the above-mentioned ingredients and water, and further excipients generally used for external preparation, such as surfactant, urea, methyl salicylate, crotamitone and menthol for the purpose of adjusting the percutaneous absorbability or improving the sense of use.

Examples of stabilizer include sodium sulfite, sodium sulfite anhydride, sodium hydrogensulfite, pyrosodium sulfite, thiosodium sulfate, and Rongalite. The content of stabilizer in the anti-inflammatory analgesic external aqueous liquid preparation is 0.02 to 1%, and more preferably 0.05 to 0.5%.

### EXAMPLES

The invention is more specifically described below by presenting examples, but it must be noted that the invention is not limited to these examples.

### Example 1

With the composition shown in Tables 1 to 3, anti-inflammatory analgesic external aqueous liquid preparation was prepared, and its properties were examined.

(Manufacturing Process)

In a part of purified water, hydroxy ethyl cellulose, hydroxypropylcellulose or a mixture of hydroxy ethylcellulose and hydroxypropyl cellulose was added and stirred, and isopropyl alcohol and diisopropyl adipate were added, then diclofenac sodium was dissolved and uniformly stirred, subsequently the pH was adjusted to about 7 by adding lactic acid, and the remaining purified water was added to a whole volume of 100 g to obtain an anti-inflammatory analgesic external aqueous liquid preparation.

The aqueous liquid preparation and the state after application was evaluated as follows.

State of Aqueous Liquid Preparation

After preparing aqueous liquid preparation, it was filled in a glass container, and its state was visually observed.

A: clear liquid

B: unclear liquid

State After Application of Aqueous Liquid Preparation

By applying 0.05 mL of aqueous liquid preparation on the upper arm by micropipette, its state was observed visually.

A: no liquid dripping

B: liquid dripping

TABLE 1

| | Product of the | | | Comparative | | |
|---|---|---|---|---|---|---|
| | invention | | | product | | |
| Component (g) | 1 | 2 | 3 | 1 | 2 | 3 |
| Diclofenac sodium | 1.00 | 1.00 | 1.00 | 1.00 | 1.00 | 1.00 |
| Diisopropyl adipate | 5.00 | 5.00 | 5.00 | 5.00 | 5.00 | — |
| Lactic acid | 0.04 | 0.04 | 0.04 | 0.04 | 0.04 | 0.04 |
| Isopropyl alcohol | 40.00 | 40.00 | 40.00 | 40.00 | 40.00 | 40.00 |
| Hydroxy ethyl | 0.05 | 0.20 | 0.45 | 0.50 | 0.04 | 0.45 |
| cellulose | | | | | | |
| Pyrosodium sulfite | 0.05 | 0.05 | 0.05 | 0.05 | 0.05 | 0.05 |
| Purified water | 53.86 | 53.71 | 53.46 | 53.41 | 53.87 | 58.46 |
| State of solvent | A | A | A | B | A | A |
| State after | A | A | A | A | B | B |
| application | | | | | | |

TABLE 2

| | |
|---|---|
| Product of | Comparative |
| the invention | product |

| Component (g) | 4 | 5 | 6 | 4 | 5 |
|---|---|---|---|---|---|
| Diclofenac sodium | 1.00 | 1.00 | 1.00 | 1.00 | 1.00 |
| Diisopropyl adipate | 5.00 | 5.00 | 5.00 | 5.00 | 5.00 |
| Lactic acid | 0.04 | 0.04 | 0.04 | 0.04 | 0.04 |
| Isopropyl alcohol | 40.00 | 40.00 | 40.00 | 40.00 | 40.00 |
| Hydroxy propyl | 0.05 | 0.20 | 0.45 | 0.50 | 0.04 |
| cellulose | | | | | |
| Pyrosodium sulfite | 0.05 | 0.05 | 0.05 | 0.05 | 0.05 |
| Purified water | 53.86 | 53.71 | 53.46 | 53.41 | 53.87 |
| State of solvent | A | A | A | B | A |
| State after | A | A | A | A | B |
| application | | | | | |

TABLE 3

| | |
|---|---|
| Product of | Comparative |
| the invention | product |

| Component (g) | 7 | 8 | 9 | 6 | 7 |
|---|---|---|---|---|---|
| Diclofenac sodium | 1.00 | 1.00 | 1.00 | 1.00 | 1.00 |
| Diisopropyl adipate | 5.00 | 5.00 | 5.00 | 5.00 | 5.00 |
| Lactic acid | 0.04 | 0.04 | 0.04 | 0.04 | 0.04 |
| Isopropyl alcohol | 40.00 | 40.00 | 40.00 | 40.00 | 40.00 |
| Hydroxy ethyl | 0.01 | 0.05 | 0.05 | 0.10 | 0.02 |
| cellulose | | | | | |
| Hydroxy propyl | 0.04 | 0.15 | 0.40 | 0.40 | 0.02 |
| cellulose | | | | | |
| Pyrosodium sulfite | 0.05 | 0.05 | 0.05 | 0.05 | 0.05 |
| Purified water | 53.86 | 53.71 | 53.46 | 53.41 | 53.87 |
| State of solvent | A | A | A | B | A |
| State after | A | A | A | A | B |
| application | | | | | |

Products 1 to 9 of the invention were clear aqueous liquid preparations, but comparative products 1, 4, and 6 exceeding 0.45 wt % in content of thickening agent were opaque liquid preparations. In comparative products 2, 5, and 7 less than 0.05% in content of thickening agent, the viscosity was low, and the liquid dripping was observed after application.

### Test Example 1

The products 1 to 9 of the invention prepared in Example 1 were filled in a tight sealed glass container, stored for 6 months at 40° C., and stability over time was observed visually. As a result, all products of the invention were free from separation of ingredients, or deposition of sediment, and were excellent in stability over time.

### Text Example 2

0.02 g each of aqueous liquid preparations of the product 2 of the invention or the comparative product 3 prepared in Example 1 were applied to the back of the human body, and in 2, 4, and 6 hours after the application, diclofenac concentration in stratum corneum (μg/cm²) was measured by HPLC method, and the percutaneous absorbability was measured. Results are shown in Table 4.

TABLE 4

| | Product 2 of | Comparative |
|---|---|---|
| Time (h) | the invention | product 3 |
| 2 | 3.3 | 0.2 |
| 4 | 3.6 | 0.1 |
| 6 | 4.1 | 0.3 |

(μg/cm²)

As compared with the comparative product 3, the product 2 of the invention was found to show a higher diclofenac concentration in stratum corneum and was excellent in percutaneous absorbability of diclofenac.

1. An anti-inflammatory analgesic external aqueous liquid preparation comprising diclofenac or its salt, a dibasic acid ester, a lower alcohol, and 0.05 to 0.45 wt % of thickening agent.

2. The anti-inflammatory analgesic external aqueous liquid preparation according to claim 1, containing 0.1 to 20 wt % of the diclofenac or its salt, 0.5 to 20 wt % of the dibasic acid ester, and 10 to 80 wt % of the lower alcohol.

3. The anti-inflammatory analgesic external aqueous liquid preparation according to claim 1 or 2, wherein the salt of diclofenac is diclofenac sodium or diclofenac ammonium.

4. The anti-inflammatory analgesic external aqueous liquid preparation according to any one of claims 1 to 3, wherein the dibasic acid ester is diisopropyl adipate, diisopropyl sebacate, or diethyl sebacate.

5. The anti-inflammatory analgesic external aqueous liquid preparation according to any one of claims 1 to 4, wherein the lower alcohol is ethyl alcohol and/or isopropyl alcohol.

6. The anti-inflammatory analgesic external aqueous liquid preparation according to any one of claims 1 to 5, wherein the thickening agent is hydroxy propyl cellulose and/or hydroxy ethyl cellulose.

7. The anti-inflammatory analgesic external aqueous liquid preparation according to any one of claims 1 to 6, wherein the pH is in the range of 5 to 8.5.
